(19) United States
(12) Patent Application Publication
(10) Pub. No.: US 2003/0033622 A1
(43) Pub. Date: Feb. 13, 2003

(54) Transgenic insect

(76) Inventors: Andrea Crisanti, London (GB); Catteruccia Flaminia, London (GB); Tony Nolan, London (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/148,772

(22) PCT Filed: Dec. 13, 2000
(86) PCT No.: PCT/GB00/04771

(30) Foreign Application Priority Data
Dec. 15, 1999 (GB) 9929681.6

(51) Int. Cl. A01K 67/033

(52) U.S. Cl. 800/13

(57) ABSTRACT

A method for the genetic modification of an insect embryo, comprises first treating an insect egg under conditions which prevent or delay the hardening of the insect egg chorion, and then injecting a transposable element into the egg to permit integration of the element into the genome of the embryo. The method permits modifications to be made to mosquitoes, which may prevent transmission of a host parasite.

### FIELD OF THE INVENTION

This invention relates to the genetic manipulation of insects. In particular, this invention relates to the genetic manipulation of mosquitos.

### BACKGROUND OF THE INVENTION

Malaria is the most important parasitic disease in the world today and is one of the major health threats in Africa, where 10% of the world's population suffers more than 90% of the world's malaria infections.

Malaria is caused by protozoan parasites of the genus Plasmodium. Of the four recognised human parasites (P. falciparum, P. vivax, P. ovale and P. malariae), P. falciparum is the most dangerous and is the major cause of mortality.

Human malaria parasites are transmitted by mosquitoes of the genus Anopheles. At least 20 of the almost 500 known types of anopheline mosquitoes have been shown to be implicated in malaria transmission. In sub-Saharan Africa, transmission is mainly caused by three anopheline species, A. gamibiae, A. arabiensis and A. funestus. These three species represent the most efficient vectorial system in the world for P. falciparum. Their distribution is limited by dry environments, salt water, low temperatures and, in the case of A. gambiae and A. arabiensis, by the thick vegetation of natural forests and humid savannah areas. These three African mosquitoes are the most efficient as malaria vectors because of their marked preference for humans as hosts as well as for their ability to adapt to human-induced environmental changes. In Asia, the most efficient malarial vector is A. Stephensi.

Control measures based on the use of pesticides have not been able to control the extremely high P. falciparum inoculation rates. Furthermore, the common occurrence of insecticide resistance, coupled with the ecological costs associated with their use, has generated the need for alternative methods to control the parasite. Attempts made by the massive distribution of antimalarial drugs have not been successful, due partly to the rapid spread of multiple drug-resistant strains of P. falciparum.

Biological control measures have been proposed as an alternative to the use of pesticides to control the spread of malaria. The production of host insects that are resistant (refractory) to the development of the parasite and thus incapable of transmitting the infection is one possible method of controlling malaria. The ability of an insect host to support the development and transmission of a parasite is called vector competence. Mosquitoes of the Culex and Aedes genera contain species that regularly feed on humans but cannot transmit malaria. The mechanisms responsible for this are various and usually species-specific. The physiology and genetic basis is incompletely known. The inability to transmit malaria could be due to the absence of some critical factor in the mosquito required by the parasite for normal development, or it could be the result of the action of some other factor(s) inhibiting parasite development.

Rosenberg et al, Insect Mol. Biol., 1985; 7:1-10, showed that A. freeborni were refractory to the simian parasite P. knowlesi because the sporozoites were unable to recognise and penetrate the mosquito salivary glands.

The identification and mapping of the genes responsible for refractoriness of a mosquito to a particular parasite is a major goal for molecular biologists. Once technologies for introducing DNA into the mosquito genome become available, the manipulation of genes determining the susceptibility or refractoriness of a given species could be of tremendous importance for preventing malaria transmission, and means for inducing refractoriness genes into wild population could then be developed (Curtis and Graves, J. Trop. Med. Hyg., 1988; 91:43-48). Furthermore, insects have various defence mechanisms, including the production of a wide variety of peptides in the body, to protect them against bacterial and fungal infections. Among the antibacterial peptides are insect defensins and cecropins, while drosomycin is the best-studied antifungal peptide. Such peptides have been shown to have the ability to interfere with the development of malaria parasites.

The lack of an established technology to transform mosquito DNA has severely hampered the attempts to control vector-borne diseases by genetic manipulation. The release of sterile males as a means of genetic control has been shown to be successful in the eradication of the screw-worm fly from North and Central America and Libya (Krafsur et al., Parasitology Today, 1987; 3:131-137). However, attempts to use this method to control mosquito populations have so far failed, mainly because of the reproductive strategies of mosquitoes, which include high fecundity, short generation time and the ability to rapidly repopulate an area after destruction of existing populations. The replacement of a mosquito population with one incapable of transmitting parasites could represent a valid alternative to the suppression of the mosquito species.

Alternatively, it may be desirable to introduce into insects foreign genes expressing anti-parasitic agents able to interfere with the life-cycle of the parasite.

For example, the use of modified Wolbachia symbionts to introduce foreign genes into Anopheles mosquitoes has been suggested (Curtis and Sinkins, Parasitology, 1998; 116 Suppl:111-115). Wolbachia represents a potentially useful gene because it is maternally inherited and causes sterility in matings of infected males to uninfected females. However, so far no data concerning mosquito transformations have been reported, due to the difficulty in introducing exogenous DNA into the mosquito genome.

Genetic manipulation of the fruitfly Drosophila has been carried out successfully using the P transposable element. Transnosable elements can be used to introduce heterologous genes into Drosophila to alter the phenotype of the insect. Other transposable elements have also been successfully introduced into the Drosophila genome, including Hobo from D. melanogaster, mariner from D. maurifiana and Minos from D. hydei (Blackman et al., EMBO J, 1989; 8:211-217; Garza et al., Genetics, 1991; 128:303-310; Loukeris et al., Proc. Natl. Acad. Sci. USA, 1995; 92:9485-9489).

The possibility of using transposable elements as DNA delivery vectors to achieve germline transformation in mosquitoes has been supported by the encouraging results obtained with Hertnes, mariner and Minos in Drosophila. However, no transposable element has been shown to be capable of transposition in anopheline mosquitoes.

The introduction of exogenous DNA into anopheline embryos represents another important limiting step in the transformation procedure. Insect embryos are surrounded by a rigid structure, the chorion, which hardens quickly after oviposition and makes the injection of DNA into anopheline embryos a very difficult and time-consuming process. A few minutes after they have been laid, eggs are already quite rigid and difficult to penetrate with commonly used needles, without killing the embryo. Survival rates of injected embryos are usually poor, and consequently large amounts of embryos need to be injected in order to obtain a significant number of survivors. Furthermore, while the chorion of Drosophila eggs is removable by bleaching, anopheline embryos are extremely sensitive to the elimination of their eggshell, which provides structural support and protection and allows gas exchange while minimising water loss.

The establishment of a reliable technology for introducing foreign genes in the Anopheles genome therefore faces two major problems: 1) the development of a DNA delivery vector capable of successful transposition in anopheline mosquitoes; and 2) the establishment of a new technology to overcome the technical difficulty of injecting DNA into mosquito embryos.

### SUMMARY OF THE INVENTION

The present invention is based, at least in part, on the realisation that injection of heterologous DNA into insect embryos can be facilitated by first manipulating the chorion to prevent or delay the hardening process. Injecting a suitable transposable element into the insect genome can then be carried out.

According to one aspect of the invention, a method for genetic modification of an insect embryo comprises the steps of:

(i) treating an insect egg under conditions which prevent or delay the hardening of the insect egg chorion; and

(ii) injecting a transposable element into the egg to permit integration of the element into the genome of the embryo.

The insect is preferably a mosquito, and more preferably an anopheline mosquito.

According to a further aspect of the invention, chorion hardening is prevented or delayed by inhibiting an enzyme involved in the hardening process. The compound p-nitrophenyl-p′-guanidinobenzoate may be used in the method of the present invention to delay the hardening of the chorion.

According to a further aspect of the invention, a genetically modified anopheline mosquito is obtainable by:

i. treating the egg of an anopheline mosquito embryo under conditions which prevents or delays the hardening of the mosquito egg chorion; and

ii. injecting a transposable element into the egg, the transposable element being capable of integrating into the genome of the mosquito embryo.

According to a further aspect of the invention, p-nitrophenyl-p′-guanidinobenzoate is used to delay the hardening of the chorion of an insect egg.

According to a further aspect, the Minos transposable element is used to transfer heterologous DNA into the genome of an anopheline mosquito embryo.

The present invention provides an efficient gene transfer technology for transforming the genome of insects, particularly anopheline mosquitoes.

This enables insects, particularly anopheline mosquitoes, to be genetically modified to exhibit particular traits or to modify the insect to prevent the spread of disease-causing parasites. The widespread applicability of this technology will be apparent to the skilled person, who may adapt existing genetic manipulations, for example as practiced on Drosophila, for use in other insects, e.g. anopheline mosquitoes.

### DESCRIPTION OF THE DRAWING

FIG. 1 illustrates the vector (MinHyg) used for transposition into a mosquito embryo. In the drawing, ActinP represents the actin5C promoter from D. melongaster; hspP represents the heat-shock promoter hsp70 from D. melongaster; hspT represents the heat-shock terminator sequence; Amp^{R} represents the ampicillin-resistance gene; Hyg^{R} represents the hygromycin-resistance gene; ML and MR represent the left and right arms of the minos transposable element, with inverted repeats represented by the black triangles; and H, E and N represent the restriction enzymes HindII, EcoRI and NotI, respectively.

### DESCRIPTION OF THE INVENTION

As stated above, an important aspect of the present invention is the treatment of the insect egg under conditions which prevent or delay the hardening of the insect egg chorion. Hardening of the chorion is mediated by a series of enzyme reactions, the first enzyme being phenol oxidase. Other enzymes include dopa decarboxylase, dopamine N-acetyl transferase and N-acetyl dopamine desaturase. Targeting these enzymes with inhibitors is one useful way of delaying or preventing the chorion hardening process. Inhibitors may be competitive or non-competitive inhibitors. Examples of inhibitors of phenol oxidase useful in the present invention, include glutathione, diethyldithiocarbamic acid, 1-phenyl-3-(2-thiazolyl)-2-thiourea and p-nitrophenyl-p′-guanidino-benzoate. Of these, p-nitrophenyl-p′-guanidinobenzoate is preferred. Other inhibitors may be apparent to the skilled person or may be identified using standard enzyme inhibition assays.

Typically, the inhibitors will be dissolved in an isotonic solution to prevent swelling of the embryos.

Amounts of inhibitor suitable for use in the invention can be determined easily. With regard to p-nitrophenyl-p′-guanidinobenzoate, a concentration of 0.1 mM has been found to be acceptable.

It may be preferable to delay (slow down) rather than prevent the hardening process. Therefore, it may be preferable to use a competitive inhibitor which can be replaced by addition of excess enzyme substrate. Alternatively, the inhibitor may be utilised over time, thereby permitting the enzyme to function with its natural substrate. Delaying hardening should be for a time sufficient for the introduction of the nucleic acid material into the egg. This may require a delay of only a few hours.

Insertion of nucleic acid into the egg may be carried out by microinjection. Methods for carrying this out will be apparent to the skilled person, using conventional apparatus.

The nucleic acid molecules may be in the form of a vector or plasmid containing a heterologous gene to be expressed in the insect embryo. Regulator sequences, including transcriptional promoters, enhancers and initiation signals, may also be present. The purpose of introducing the nucleic acid molecules may be to produce a transgenic insect, having particular genetic traits. Technology for the production of transgenic animals and insects are known and may be adapted for use in the present invention.

The nucleic acid is integrated into the insect genome using transposable elements. Integration (transposition) is often facilitated by the enzyme transposase, and the transposable element often comprises inverted repeats which function to direct the transposase to the correct position, to initiate excision. Genetic constructs, comprising a transposable element combined (in a genetic fusion) with a heterologous gene, may be prepared using conventional technology, and inserted into the insect egg to produce a transgenic insect.

In addition to the heterologous gene, the transposable element may comprise the regulatory factors that ensure successful expression can occur.

Transposable elements useful in the present invention may be identified based on experiments carried out on other organisms, e.g. in Drosophila. For example, Hermes from Musca domestics (Atkinson et al., Proc. Natl. Acad. Sci. USA, 1993; 90:9693-9697) is able to transpose in embryos of Drosophila melongaster. Mariner from D. mauritania (Haymer and Marsh, Dev. Genet., 1986; 6:281-291) was shown to transpose in Bactrocera tryoni.

A preferred transposable element is Minos, found in Drosophila hydei (Franz and Savakis, Nucleic Acids Res., 1991; 19: 6646). It has now been found that minos transposase can mediate precise insertions into the genome of Anopheles mosquitoes and permit interplasmid transposition to occur. Therefore, in a preferred embodiment, the invention may be carried out using a Minos transposable element to integrate a heterologous nucleic acid molecule into the genome of an insect embryo, preferably in the presence of a minos transposase. The transposable element may be in the form of a plasmid vector together with a foreign gene and further comprising regulatory sequences, e.g. a promoter. In a preferred embodiment, the promoter is the actin5c promoter from D. melongaster. In a further preferred embodiment, the minos transposase gene is located on a separate helper plasmid, for separate introduction into the embryo.

The transposable element may be used to integrate into the insect embryo a heterologous gene which can be expressed in vivo. Alternatively, integration of the transposable element may be required to integrate a heterologous polynucleotide which can be used to disrupt expression of a particular gene. For example, an RNA molecule may be used for gene silencing.

The heterologous gene may be used to control the transmission of a parasite, e.g. plasmodium. For example, the gene may encode a product that protects the insect from infection or which encodes an anti-parasitic agent, able to interfere with the life-cycle of the parasite. Some antibacterial peptides are known, including defensins, which may be of use. Alternatively, the gene may be used to produce sterile males which may be released as a means of genetic control. The use of a sex-specific promoter has been proposed for use in Drosophila (Thomas et al., Science, 2000; 287(5462): 2474-2476), and may be used in the present invention. The Wolbachia gene may also be used. Suicide genes may also be introduced which can be activated by exposure to certain chemicals. Other suitable genes will be apparent to the skilled person.

The transposable elements may also be of use in assays for identifying compounds or products that have insecticidal activity, or for mapping genes responsible for refractoriness of, for example, mosquitoes, to a particular parasite. The insertion of foreign or heterologous genes into a genome can be used to identify enhancer elements located in the genome. Significant levels of the product of the gene will not be detectable unless the transposable element inserts next to a region containing the enhancer element. The transposable elements may also be used to perform in vivo site-directed mutagenesis, as described in Banga and Boyd, Proc. Natl. Acad. Sci. USA, 1992; 89:1735-1739.

The following Example illustrates the invention.

### EXAMPLE

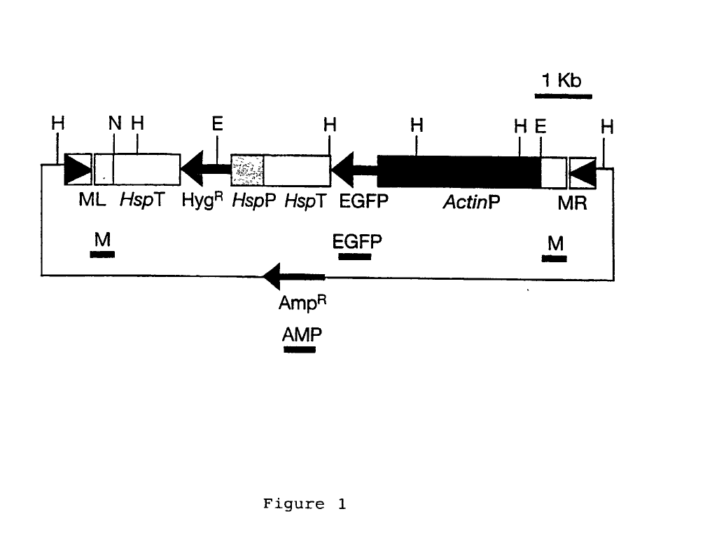

In the following experiment, the plasmid vector termed MinHyg (illustrated in FIG. 1), was used to achieve integration of a heterologous gene into the genome of an anopheline mosquito. As shown in FIG. 1, the green fluorescent protein gene, GFPS65T (GFP) (Heim et al., Nature, 1995; 373:663-664) was chosen as the reporter gene, to show that successful integration of DNA had been achieved.

The actin promoter from the D. melanogaster actin5C gene was chosen to drive the expression of the GFPS65T marker (Fyrberg et al., Cell, 1983; 33:115-123).

The hygromycin gene, under the control of the inducible heat-shock protein 70 (hsp70) promoter, was also incorporated into the vector to act as a selectable marker in the event that selection with GFP could not be achieved.

The experiment was performed as follows. Blood fed A. Stephensi mosquitoes were allowed to lay eggs 48-72 hours after a blood meal. Eggs were laid in a petri dish containing 3 mm paper soaked in a p-nitrophenyl-p′-guanidinobenzoate (NPGB) solution (Sigma cat. N 8010) 0.1 mM in isotonic buffer (150 mM NaCl, 5 mM KCl, 10 mM HEPES, 2.5 MM CaCl₂, pH 7.2). NPGB is not soluble in water; it was first dissolved in DMSO and then isotonic buffer was added to make the 0.1 mM final solution. The use of the isotonic buffer is essential as it prevents the embryos from swelling. The petri dish was removed from the mosquito cage 30 minutes after the first oviposition had occurred. Eggs were then left in NPGB until injection, which was carried out between 90 and 120 minutes after oviposition. A total of around 30 embryos were placed on a glass slide covered with paper wet with isotonic buffer, with their posterior poles aligned and oriented towards the inner part of the glass slide. As soon as the embryos started drying they were transferred, by applying a gentle pressure, onto another slide on which a strip of double-sided tape had been stuck at one end. The embryos were then covered with water-saturated halocarbon oil to prevent further desiccation.

Glass needles (Eppendorf Femtotips) were loaded with 2 μl of the DNA solution by using microloader tips (Eppendorf). The embryos were microinjected with a mixture of 100 μg/ml of the helper intronless plasmid pHSS6hsILMi20 (Klinakis et al., Insect Mol. Biol. 2000; 9(3) :269-275) and plasmid MinHyg(400 μg/ml). The helper plasmid provides the transposase activity necessary for Minos transposition, while plasmid MinHyg contains the GFP cloned within the inverted terminal repeats of Minos. Microinjections is were performed by using an Eppendorf transjector 5246 micromanipulator at 10× magnification. The needle was introduced into the posterior pole of the embryos at a 150 angle. The injected volume was controlled by regulating the injection pressure and time. After injection, the embryos were removed gently from the halocarbon oil with the help of a brush and transferred into a new petri dish containing a stacked layer of filter paper soaked with isotonic buffer to prevent the eggs from floating. They were then allowed to hatch. Hatched larvae were then analysed under the UV light to detect GFP expression.

An average of 29% of injected embryos survived and around 50% of the hatched larvae showed strong transient expression of GFP, as monitored by fluorescence. Survival to adult stage (G₀) averaged 10% and was a good predictor of successful transformation. In two experiments that gave 16% adult survival, the progeny of 69 G₀ mosquitoes yielded 92 fluorescent individuals among the 10,539 G₁ larvae analysed. It was subsequently determined that the 92 fluorescent G₁ individuals were derived from a minimum of five independent G₀ founders, representing a transformation frequency of 7% (5/69 surviving adults). This frequency is higher than that reported in D. melanogaster and C. capitata transformation experiments using Minos marked with the white gene marker (Loukeris et al, Science, 1995; 270: 2002-2005, and Proc. Natl. Acad. Sci. USA, 1995; 92: 9485-9489).

These successful experiments provide, for the first time, compelling evidence that germline transformation of anopheline mosquitoes is feasible and that Minos represents an excellent candidate for its achievement.

1. A method for the genetic modification of an insect embryo, comprising the steps of:
i. treating an insect egg under conditions which prevent or delay the hardening of the insect egg chorion; and
ii. injecting a transposable element into the egg to permit integration of the element into the genome of the embryo.

2. A method according to claim 1, wherein the insect is an anopheline mosquito.

3. A method according to claim 2, wherein the mosquito is A. gambiae, A. arabiensis or A. stephensi.

4. A method according to any preceding claim, wherein the transposable element is minos.

5. A method according to any preceding claim, further comprising the injection into the egg of a vector comprising a transposase gene, capable of expression in vivo.

6. A method according to any preceding claim, wherein the transposable element comprises a heterologous gene that is capable of being expressed after integration into the embryo.

7. A method according to claim 6, wherein the heterologous gene encodes a product that prevents transmission of a host parasite.

8. A method according to claim 7, wherein the product is an anti-bacterial agent.

9. A method according to claim 7 or claim 8, wherein the host parasite is Plasmodium falciparum.

10. A method according to claim 6, wherein the gene is a suicide gene.

11. A method according to claim 6, wherein the gene product causes male sterility.

12. A method according to any preceding claim, wherein chorion hardening is prevented or delayed by inhibiting an enzyme involved in chorion hardening.

13. A method according to claim 12, wherein the enzyme is phenol oxidase.

14. A method according to claim 12 or claim 13, wherein the inhibitor is p-nitrophenyl-p′-guanidinobenzoate.

15. A genetically modified anopheline mosquito, obtainable by:
1. treating the egg of an anopheline mosquito embryo under conditions which prevent or delay the hardening of the mosquito egg chorion; and
ii. injecting the transposable element Minos into the egg, the transposable element being capable of integrating into the genome of the mosquito embryo.

16. A genetically modified anopheline mosquito obtainable by:
i. treating the egg of an anopheline mosquito embryo under conditions which prevent or delay the hardening of the mosquito egg chorion; and
ii. injecting a transposable element into the egg, the transposable element being capable of integrating into the genome of the mosquito embryo,
wherein the transposable element comprises a heterologous gene as defined in any of claims 7 to 11.

17. Use of an inhibitor of an enzyme involved in the process of chorion hardening, to prevent or delay hardening of the chorion.

18. Use according to claim 17, wherein the enzyme is phenol oxidase.

19. Use according to claim 17 or claim 18, wherein the inhibitor is p-nitrophenyl-p′-guanidinobenzoate.

20. Use of the Minos transposable element to transfer a heterologous gene into the genome of an anopheline mosquito embryo.

21. Use according to claim 20, wherein the heterologous gene is as defined in any of claims 6 to 11.
